(19) United States
(12) Patent Application Publication
(10) Pub. No.: US 2008/0249329 A1
(43) Pub. Date: Oct. 9, 2008

(54) Process For Esterification Of An Organic Acid

(75) Inventors: Wilhelmus Hubertus Joseph Boesten, Sittard (NL); Dennis Heemskerk, Schinveld (NL)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., TE Heerlen (NL)

(21) Appl. No.: 12/088,159

(22) PCT Filed: Sep. 26, 2006
(86) PCT No.: PCT/EP2006/066756

§ 371 Date: Jun. 6, 2008

(30) Foreign Application Priority Data
Sep. 29, 2005 (EP) 05108997.7

(51) Int. Cl. C07C 229/52

(52) U.S. Cl. 560/19

(57) ABSTRACT

The invention relates to a sulphonic acid salt of an amino acid alkyl ester The invention further relates to a process for the esterification of an organic acid into the corresponding organic acid ester comprising bringing the organic acid into contact with a strong acid and a solution comprising dialkylcarbonate in a reaction mixture. The invention further relates to the use of a sulphonic acid salt of an amino acid alkyl ester in the synthesis of a β-lactam antibiotic.

The present invention relates to a sulphonic acid salt of an amino acid alkyl ester, a process for the esterification of an organic acid with dialkylcarbonate, and the use of a sulphonic acid salt of an amino acid alkyl ester.

An alkanesulphonic acid salt of a ω-benzyl ester of amino dicarboxylic acid is known from U.S. Ser. No. 04/0133033, in particular methanesulphonic acid salt of ω-benzyl ester of glutamic acid and aspartic acid. The alkanesulphonic acid salt of a ω-benzyl ester of amino dicarboxylic acid in U.S. Ser. No. 04/0133033 was prepared by acid esterification by reacting the amino dicarboxylic acid with a benzyl alcohol in the presence of an alkane sulphonic acid.

A p-toluene sulphonic acid salt from phenylglycine ethyl ester and isopropyl ester is known from L. Duhamel & J.-C. Plaquevent, Bull. Soc. Chim, 1982, p. 75-83, wherein these compounds were prepared by acid esterification in the presence of benzene and ethanol or isopropanol, respectively.

It was found that the conversion of an amino acid into its corresponding ester in the esterification process as disclosed in U.S. Ser. No. 04/0133033 and L. Duhamel & J.-C. Plaquevent, Bull. Soc. Chim, 1982, p. 75-83 was relatively low due to water formation in the esterification process.

The aim of the present invention is the provision of an alternative sulphonic acid salt of an amino acid alkyl ester, which can be obtained in a sufficiently high conversion. The aim is achieved with a sulphonic acid salt of an amino acid alkyl ester, according to the present invention.

Surprisingly, the sulphonic acid salt of an amino acid alkyl ester was advantageously obtained in the process for the esterification according to the present invention in a high conversion and without the formation of water.

As used herein, the amino acid in the sulphonic acid salt of the amino acid alkyl ester according to the invention may be any suitable aliphatic or aromatic amino acid. A suitable amino acid may for example be an amino acid selected from the group consisting of dihydro-phenylglycine and phenylglycine.

It was found that when a salt, for instance a HCl salt, of dihydro-phenylglycine alkyl ester or of phenylglycine alkyl ester is used as activated side chain in the enzymatic synthesis of a β-lactam antibiotic, for instance cephradine, or cephalexin,

cefaclor, and ampicillin respectively, the acylase used in the acylation reaction may be inhibited by side products present in the HCl salt of dihydro-phenylglycine alkyl ester or of phenylglycine alkyl ester.

Surprisingly, it was found that a sulphonic acid salt of dihydro-phenylglycine alkyl ester and of phenylglycine alkyl ester, according to the present invention does not comprise side products which inhibit the acylase used in the enzymatic acylation reaction in the synthesis of β-lactam antibiotics.

A suitable amino acid in the sulphonic acid salt of an amino acid alkyl ester according to the present invention may also be phenylalanine, α-methyl-phenylglycine, β-phenylalanine, for instance, (L)-phenyl alanine, (D)-α-methyl-phenylglycine, ε-amino-capronic acid, or (L)-β-phenylalanine (3-amino-3-phenyl-propionic acid).

A sulphonic acid salt of an ε-amino-capronic acid alkyl ester may for instance be used in the synthesis of caprolactam.

The amino acid alkyl ester in the sulphonic acid salt according to the present invention may be present in any enantiomeric form, such as in the form of the pure (D)-enantiomer or the pure (L)-enantiomer or in the form of a racemic mixture. When the sulphonic acid salt of an amino acid alkyl ester, eg. phenylglycine alkyl ester or dihydro-phenylglycine alkyl ester, is to be used in the synthesis of a β-lactam antibiotic, preferably the amino acid alkyl ester is present in the form of the (D)-enantiomer.

Alternatively, the amino acid alkyl ester may be present in the form of the (L)-enantiomer. A sulphonic acid salt of (L)-phenylalanine alkyl ester, may for instance be used in the synthesis of aspartame.

The amino acid alkyl ester may also be present in the form of a racemic mixture.

The alkyl in the sulphonic acid salt of an amino acid alkyl ester may comprise any suitable number of carbon atoms. Preferably, the alkyl comprises 1 to 20 carbon atoms, preferably 1 to 15 carbon atoms, more preferably 1 to 10 carbon atoms, more preferably 1 to 6 carbon atoms. The alkyl in the sulphonic acid salt of amino acid alkyl ester may for example be methyl, ethyl, propyl, isopropyl, butyl, or isobutyl, pentyl, isopentyl, hexyl or isohexyl. Preferably, the alkyl in the sulphonic acid salt of an amino acid alkyl ester according to the invention is methyl or ethyl.

The sulphonic acid in the sulphonic acid salt according to the invention may be an alkane sulphonic acid of the formula R—SO₃H, for instance a methane sulphonic acid (R═CH₃) or an aryl sulphonic acid of the formula R—SO₃H, for instance p-toluene sulphonic acid (R═C₇H₈), or benzene sulphonic acid (R═C₆H₆) or may be sulphuric acid (H₂SO₄). Preferably, the sulphonic acid is methane sulphonic acid.

Preferably, the sulphonic acid salt of an amino acid alkyl ester is not a methane sulphonic acid salt of o)-benzyl ester of an amino dicarboxylic acid.

Preferably, the sulphonic acid salt of an amino acid alkyl ester is not a p-toluene sulphonic acid salt selected from the group consisting of phenyl glycine ethyl ester and phenyl glycine isopropyl ester.

The present invention also relates to a process for the esterification of an organic acid with dialkylcarbonate.

U.S. Ser. No. 04/0133033 discloses a process for the preparation of ω-benzyl esters of amino dicarboxylic acid wherein an amino dicarboxylic acid is esterified with a benzyl alcohol in the presence of an alkanesulphonic acid. A disadvantage of the esterification process in US 04/0133033 is that one mole water is formed per mole of ω-benzyl alcohol, which suppresses the maximum conversion of amino dicarboxylic acid into the corresponding ester that can be achieved. Water will react with the formed ester bond, which shifts the equilibrium to the original reactants, i.e. the amino dicarboxylic acid and benzyl alcohol resulting in a decreased conversion.

The aim of the present invention is the provision of an alternative process for the esterification of an organic acid into the corresponding organic acid ester, wherein no water is formed, and which results in an increased conversion of the organic acid into its corresponding ester than disclosed in the prior art.

The aim is achieved according to the invention by a process for the esterification of an organic acid into the corresponding organic acid ester comprising bringing the organic acid into contact with a strong acid and a solution comprising dialkylcarbonate in a reaction mixture.

As used herein the organic acid may be an amino acid or a carboxylic acid.

It is known that an amino acid can be esterified with a dialkylcarbonate under alkaline conditions. Under alkaline conditions however, the amino group of the amino acid is a strong nucleophile, which easily attacks the electrophile of dialkylcarbonate. This results in the formation of side-products such as carbamate, diketopiperazine (DKP), dipeptide, or polypeptide. In addition, under alkaline conditions racemisation of the amino acid and amino acid ester easily occurs.

It was surprisingly found that a high conversion of organic acid into its corresponding organic acid ester could be achieved and that no side products are formed in the esterification process according to the present invention.

Other advantages of the process according to the present invention are the following:

The initial water formed in the process for the esterification according to the present invention, immediately reacts with dialkylcarbonate to form carbondioxide and the corresponding alcohol. Therefore, the initial formed water cannot react with the ester bond in the amino acid alkyl ester.

In addition, no racemisation of the organic acid and the corresponding organic acid alkylester occurs.

Likewise, when the organic acid ester is an amino acid, no reaction was found to take place between the amino group and dialkylcarbonate.

It was also found that esterification of an organic acid with a dialkylcarbonate under acid conditions according to the present invention resulted in an organic acid alkyl ester, which could be easily isolated from the reaction mixture.

The organic acid in the process according to the invention may be any suitable organic acid that may be esterified with a dialkylcarbonate. Preferably, the organic acid is any suitable aliphatic or aromatic amino acid or any suitable aliphatic or aromatic carboxylic acid. The organic acid to be esterified may for example be an amino acid such as phenylalanine, α-methyl-phenylglycine, β-phenylalanine, phenylglycine, dihydro-phenylglycine or ε-amino-capronic acid, for instance (L)-phenylalanine, (D)-α-methyl-phenylglycine, (L)-β-phenylalanine, (D)-phenylglycine and (D)-dihydro-phenylglycine.

A suitable organic acid that may be esterified with a dialkylcarbonate in the process according to the present invention may also be a carboxylic acid, for example phenylacetic acid or benzoic acid.

The organic acid and the corresponding organic acid ester in the process according to the present invention may be present in any suitable enantiomeric form, such as the D-enantiomer, the L-enantiomer or in the form of a racemic mixture.

In the process for the esterification according to the present invention, the organic acid, the strong acid and the dialkylcarbonate may be brought into contact with each other in any order and within any suitable period of time, depending on the stirrability of the reaction mixture, which is, amongst others, dependent on the concentration of the reactants, i.e. organic acid, dialkylcarbonate and strong acid in the reaction mixture. For instance, the organic acid may be brought into contact at once with the strong acid and the solution comprising dialkylcarbonate at the start of the reaction. The organic acid may also be brought into contact with the strong acid and the solution comprising dialkylcarbonate by dosing the strong acid during a period of time of between 1 to 120 min, preferably during a period of time of 5 to 90 min, preferably during a period of time of 10 to 60 min.

Any strong acid which is suitable to acidify the reaction mixture in the process according to the invention may be used in the esterification process. As defined herein, a strong acid is an acid having an acid dissociation constant (pK) smaller than or equal to (≦) 1. A suitable strong acid is for instance methane sulphonic acid (CH₃—SO₃H), p-toluene sulphonic acid (C₇H₈—SO₃H), benzene sulphonic acid (C₆H₆—SO₃H) or sulphuric acid (H₂SO₄). Preferably, methane sulphonic acid is used as strong acid in the process according to the present invention.

In case that the organic acid is an amino acid it is preferred that the strong acid in the reaction mixture is present in an amount equal to or larger than equimolar to the amino acid. In case that the organic acid is a carboxylic acid it is preferred that the strong acid in the reaction mixture is present in an amount catalytic to or larger than the amount of carboxylic acid.

The solution comprising dialkylcarbonate in the esterification process according to the present invention may additionally comprise an alcohol. In the case that the organic acid is an amino acid in the process for the esterification according to the present invention, it was advantageously found that when an alcohol is present in the solution comprising dialkylcarbonate, a higher amount of amino acid salt was dissolved in the reaction mixture and the esterification reaction proceeded faster. The amino acid salt is formed when it is brought into contact with the strong acid in the reaction mixture prior to the esterification in the process according to the present invention.

When an alcohol is present in the solution comprising dialkylcarbonate, it is essential that the alcohol comprises an identical number of carbon atoms as the number of carbon atoms of the alkyl in dialkylcarbonate. For example, when alkyl in dialkylcarbonate is methyl or ethyl, the alcohol is methanol and ethanol, respectively.

The alkyl in dialkylcarbonate in the process according to the present invention may comprise any number of carbon atoms, for instance between 1 to 20 carbon atoms, preferably between 1 to 15, more preferably between 1 and 10 carbon atoms. Preferably, the alkyl in dialkylcarbonate comprises 1 to 6 carbon atoms. The alkyl may for example be methyl, ethyl, n-propyl, isopropyl, n-butyl, iso-butyl, pentyl, isopentyl, hexyl or isohexyl. Preferably, the alkyl is methyl or ethyl.

The esterification may be carried out at any suitable temperature in combination with suitable pressure and during any suitable period of time. In one embodiment, the esterification is carried out at atmospheric pressure and at a suitable temperature and during a suitable period of time. In another preferred embodiment, the esterification is carried out at a pressure above atmospheric, for instance at a pressure equal to or higher than 1 kg/m², more preferably at a pressure equal to or higher than 2 kg/m², even more preferably at a pressure equal to or higher than 3 kg/m². Since the reflux temperature of the reaction mixture is increased at a pressure above atmospheric, the reaction temperature of the esterification may be increased accordingly. The advantage of using an increased temperature at elevated pressure is that reaction time is decreased.

A pressure above atmospheric may be build up in a closed esterification reactor by the carbon dioxide (CO₂) which is produced as a result of the reaction of the water, liberated during the esterification reaction, with the dialkylcarbonate. Alternatively, the overpressure may be established in the closed esterification reactor by applying an overpressure using an external inert gas such as nitrogen (N₂), argon (Ar), etceteras. The esterification may for example comprise keeping the reaction mixture at a temperature below the reflux temperature of the dialkylcarbonate for at least 1 hour, preferably for at least 4 hours. Depending on the dialkylcarbonate, it was found that when the reaction mixture was kept at a temperature of below the reflux temperature of the dialkylcarbonate for a certain period of time at the start of the esterification, the reaction mixture remained stirrable. This was found to be in particularly advantageous when the concentration of organic acid in the reaction mixture is high in the case the organic acid is an amino acid. A high concentration of the amino acid may be between 10 and 50% w/w, for instance 20 to 40% or 25 to 35% w/w.

The reflux temperature is dependent on the dialkylcarbonate in the reaction mixture, and may suitably be below 90° C. It was found that by keeping the temperature of below about 90° C., racemization of an optically active organic acid, or organic acid alkylester did not occur.

As defined herein the reflux temperature is the boiling temperature of the dialkylcarbonate used in the esterification process.

In another preferred embodiment the esterification process comprises keeping the reaction mixture at a temperature of at least the reflux temperature of the dialkylcarbonate for at least 6 hours, preferably at least 24 hours, preferably between 48 hours and 7 days. It was found that when the temperature in the reaction mixture was kept at at least the reflux temperature of the dialkylcarbonate, the esterification reaction rate was increased.

During the esterification of the organic acid, an organic acid ester is formed. The organic acid ester may be present in dissolved form or may crystallise during esterification of the organic acid.

In a preferred embodiment of the present invention the organic acid ester is isolated from the reaction mixture.

Isolation of the organic acid ester from the reaction mixture may be performed in any suitable way. Preferably, isolation of the organic acid ester comprises distilling off part of the dialkylcarbonate or dialkylcarbonate and alcohol present in the reaction mixture. It was found that distilling off part of the dialkylcarbonate or dialkylcarbonate and alcohol present in the reaction mixture resulted in an improved yield of the organic acid ester.

Preferably, isolation of the organic acid ester also comprises adding fresh dialkylcarbonate to the reaction mixture, wherein the alkyl in the fresh dialkylcarbonate has the same number of carbon atoms as in the dialkylcarbonate used in the esterification process. Preferably, fresh dialkylcarbonate is added to the reaction mixture subsequent to distilling off part of the dialkylcarbonate or dialkylcarbonate and alcohol from the reaction mixture.

Isolation of the organic acid ester from the reaction mixture may be performed at any suitable temperature. The organic acid ester may for example be isolated at a temperature below the temperature at which the organic acid crystallises. Preferably, the organic acid ester is isolated at a temperature below 50° C., preferably below 40° C., more preferably below 35° C., more preferably below 30° C.

The isolation of the organic acid ester may also comprise adding a base to the reaction mixture, which neutralises the excess of strong acid present in the reaction mixture. Any organic or inorganic base may be used to neutralise the reaction mixture. Examples of suitable organic bases are triethylamine, diethylamine, diisopropylethylamine, and dicyclohexylamine. An inorganic base may for example be ammonia (NH₃) in gaseous form or in solution. Preferably, triethylamine is used to neutralise the reaction mixture.

The organic acid ester may be isolated from the reaction mixture in any suitable form, preferably in crystalline form. When the organic acid ester is an amino acid ester, the amino acid ester is preferably crystallised in the form of a salt. For instance, the amino acid ester may be crystallised in the form of a sulphonic acid salt when a sulphonic acid is used as the strong acid in the esterfication process according to the present invention.

The organic acid ester in crystalline form may be further isolated from the reaction mixture by known methods in art, such as centrifugation and filtration.

In another preferred embodiment the organic acid ester in crystalline form may be dried at any suitable temperature and under any suitable pressure, for instance by drying under vacuum.

The present invention further relates to the use of a sulphonic acid salt of an amino acid alkyl ester in the synthesis of a β-lactam antibiotic.

The sulphonic acid salt of an amino acid alkyl ester may be used as activated side chain in the enzymatic synthesis of a β-lactam antibiotic, wherein the activated side chain may be enzymatically coupled to a suitable β-lactam nucleus in the presence of an acylase, for instance as described in WO 2005/003367, WO00/00201, or EP 0771357.

A suitable β-lactam nucleus is for instance 6-amino penicillanic acid (6-APA) 7-aminodesacetoxy cephalosporanic acid (7-ADCA), 7-amino cephalosporanic acid (7-ACA), 7-amino-3-[(Z)-1-propenyl]-3-(desacetoxymethyl)cephalosporanic acid, or 7-amino-3-chloro-cephalosporanic acid (7-ACCA).

In a preferred embodiment, the invention relates to the use of a sulphonic acid salt of (D)-phenylglycine alkyl ester in the synthesis of a β-lactam antibiotic selected from the group consisting of cephalexin, cefaclor and ampicillin.

In another preferred embodiment, the invention relates to the use of a sulphonic acid salt of (D)-dihydro-phenylglycine alkyl ester in the synthesis of cephradine.

The alkyl in the use of a sulphonic acid salt of an amino acid alkyl ester in the synthesis of a β-lactam antibiotic, may comprise any number of carbon atoms, for instance between 1 to 20 carbon atoms, preferably between 1 to 15, more preferably between 1 to 10 carbon atoms. Preferably, the alkyl in the use of a sulphonic acid salt of an amino acid alkyl ester in the synthesis of a β-lactam antibiotic comprises 1 to 6 carbon atoms. The alkyl may be methyl, ethyl, n-propyl, isopropyl, n-butyl, isobutyl, pentyl, isopentyl, hexyl or isohexyl. Preferably, the alkyl is methyl or ethyl.

The sulphonic acid in the use of a sulphonic acid salt of an amino acid alkyl ester in the synthesis of a β-lactam antibiotic may be an alkane sulphonic acid of the formula R—SO₃H, for instance a methane sulphonic acid (R═CH₃) or an aryl sulphonic acid of the formula R—SO₃H, for instance p-toluene sulphonic acid (R═C₇H₈), or benzene sulphonic acid (R═C₆H₆) or may be sulphuric acid (H₂SO₄). Preferably, the sulphonic acid is methane sulphonic acid.

The following examples are for illustrative purposes only and are not to be construed as being limited thereto.

### EXAMPLES

### Example 1

### Preparation of (D)-dihydro-phenylglycine methyl ester methanesulphonic acid salt (DHME.CH₃SO₃H)

0.5 mol (76.6 g) (D)-dihydro-phenylglycine (ee_{D}>99%) was suspended in a solution of 300 ml dimethylcarbonate and 10 ml methanol. Subsequently, 0.7 molmethanesulphonic acid was dosed to the suspension in 30 to 60 min at a temperature increasing from 30 to 75° C. After all the methane sulphonic acid was dosed to the reaction mixture, the temperature was kept at the reflux temperature of dimethylcarbonate, i.e. about 84 to 85° C. The temperature was kept for 5 hours at 84 to 85° C., after which the esterification reaction proceeded for 24 hours at a temperature of 82° C. The conversion of dihydro-phenylglycine into dihydro-phenylglycine methyl ester after 24 hours at 82° C. was 99.5%. Subsequently, 50 ml of methanol/dimethylcarbonate was distilled off from the reaction mixture at T=82° C. at atmospheric pressure. Thereafter, 150 ml of dimethylcarbonate was added to the reaction mixture and the reaction mixture was slowly cooled to a temperature of about 20° C. An amount of 0.17 moles of triethylamine was dosed to the reaction mixture in 30 min at 20° C. Subsequently, the reaction mixture was stirred at 20° C. for 30 min. The reaction mixture was filtered by G3 filtration and the residue was washed 4 times with 50 ml dimethylcarbonate. The residue consisting of dihydro-phenylglycine methyl ester methanesulphonic acid salt (DHME.CH₃SO₃H) was dried under vacuum at 60° C. under nitrogen, for 1 hour.

The yield of (D)-dihydro-phenylglycine methyl ester was 114.5 g (87%; ee_{D}≧99%).

The melting point of DHME.CH₃SO₃H was 130° C. as determined with a Buchi 535 melting point apparatus. FIG. 1 shows the IR spectrum of DHME.CH₃SO₃H. The IR spectrum was determined with a Perkin Elmer Spectrum One.

### Example 2

### Preparation of (D)-phenylglycine methyl ester methanesulphonic acid salt (PGM.CH₃SO₃H)

0.5 mol (75.6 g) (D)-phenylglycine (ee_{D}>99) was suspended in a solution of 300 ml dimethylcarbonate and 10 ml methanol. Subsequently, 0.7 mol methanesulphonic acid was dosed to the suspension in 30 to 60 min at a temperature increasing from 30 to 75° C. After all the methane sulphonic acid was dosed to the reaction mixture, the temperature was kept at the reflux temperature of dimethylcarbonate, i.e. 84 to 85° C., for 1 hour. The esterification reaction was proceeded for 26 hours at a temperature of 82° C. The conversion of dihydrophenylglycine into dihydrophenylglycine methyl ester after 26 hours at 82° C. was 99.3%.

Subsequently, 100 ml of methanol/dimethylcarbonate was distilled off from the reaction mixture at T=82° C. at atmospheric pressure. Thereafter, 200 ml of dimethylcarbonate was added to the reaction mixture and the reaction mixture was slowly cooled down to a temperature of about 20° C. An amount of 0.17 moles of triethylamine was dosed to the reaction mixture in 30 min at 20° C. Subsequently, the reaction mixture was stirred at 20° C. for 30 min. The reaction mixture was filtered by G3 filtration and the residue was washed 4 times with 50 ml dimethylcarbonate. The residue consisting of phenylglycine methyl ester methanesulphonic acid salt (PGM.CH₃SO₃H) was dried under vacuum at 60° C. under nitrogen atmosphere, for 1 hour.

The yield of (D)-phenylglycine methyl ester was 124 g (95%; ee_{D}>99%).

The melting point of PGM.CH₃SO₃H was 156° C. as determined with a Buchi 535 melting point apparatus.

FIG. 2 shows the IR spectrum of PGM.CH₃SO₃H. The IR spectrum was determined with a Perkin Elmer Spectrum One.

### Example 3

### Preparation of (D)-phenylglycine methyl ester sulphuric acid salt (PGM.H₂SO₄)

0.5 mole (75.6 g) (D)-phenylglycine (ee_{D}>99) was suspended in a solution of 250 ml dimethylcarbonate. Subsequently, 0.6 mol H₂SO₄ (sulphuric acid) was dosed to the suspension in 30 to 60 min at a temperature increasing from 80 to 90° C. The esterification reaction was proceeded for 20 hours at a temperature of 83° C. The conversion of phenylglycine into phenylglycine methyl ester after 20 hours at 83° C. was >99%.

### Example 4

### Preparation of (L)-phenylalanine methyl ester methanesulphonic acid salt

0.25 (41.3 g) mol of (L)-phenylalanine was suspended into a solution comprising 150 ml dimethylcarbonate and 5 ml methanol. Subsequently, 0.35 mol of methanesulphonic acid was dosed to the reaction mixture at T=20° C. increasing to T=84° C., within 1 hour. The esterification reaction was proceeded for 24 hour, after which the conversion into (L)-phenylalanine methyl ester was 99.4%. The reaction mixture was cooled to T=25° C. and the reaction mixture was subjected to a G3 filtration. The residue was washed 4 times with 25 ml of dimethylcarbonate. The residue was dried under vacuum at 60° C. under nitrogen atmosphere, for 1 hour.

### Example 5

### Preparation of 3-amino-3-phenylpropionic acid methyl ester methanesulphonic acid salt

0.25 mol of 3-amino-3-phenylpropionic acid was suspended into a solution comprising 150 ml dimethylcarbonate and 5 ml methanol. Subsequently, 0.35 mol of methanesulphonic acid was dosed to the reaction mixture at a temperature increasing from 30° C. to 60° C. within 15 min. 20 ml of methanol and 40 ml of dimethylcarbonate was added to the reaction mixture and the reaction mixture was kept for 12 hour at 30° C. The esterification reaction was proceeded for 24 hour at a temperature slowly increasing to 70° C. after which the conversion into 3-amino-3-phenylpropionic acid methyl ester was 99.4%. Subsequently, 70 ml of methanol/dimethylcarbonate was distilled off at 80° C. and the reaction was proceeded at 84 to 85° C. The reaction mixture was cooled to T=25° C. and the reaction mixture was subjected to a G3 filtration. The residue was washed 4 times with 25 ml of dimethylcarbonate. The residue was dried under vacuum at 60° C. under nitrogen atmosphere, for 1 hour.

### Example 6

### Preparation of (D)-α-methyl-phenylglycine methyl ester methanesulphonic acid salt

0.25 ml (41.3 g) (D)-α-methyl-phenylglycine was suspended into a solution comprising 150 ml dimethylcarbonate and 5 ml methanol. Subsequently, 0.35 mol of methanesulphonic acid was dosed to the reaction mixture at T=20° C. increasing to T=84° C., within 1 hour. The esterification reaction was proceeded for 48 hour, after which the conversion into D-α-methyl-phenylglycine methyl ester was 97%. The reaction mixture was cooled to T=25° C. and kept at 25° C. for 24 hours. Crystallisation was initiated, and subsequently the reaction mixture was subjected to a G3 filtration. The residue was washed 4 times with 25 ml of dimethylcarbonate. The residue was dried under vacuum at 30° C. under nitrogen atmosphere, for 1 hour.

### Example 7

### Preparation of ε-amino-capronic acid methyl ester methanesulphonic acid salt

0.25 ml (41.3 g) ε-amino-capronic acid was suspended into a solution comprising 150 ml dimethylcarbonate and 5 ml methanol. Subsequently, 0.35 mol of methanesulphonic acid was dosed to the reaction mixture at T=20° C. increasing to T=84° C., within 20 min. The esterification reaction was proceeded for 20 hours at 84 to 85° C., after which the conversion into ε-amino-capronic acid methyl ester was >99%. The reaction mixture was cooled to T=20° C. Crystallisation started at 35° C. Subsequently the reaction mixture was subjected to a G3 filtration. The residue was washed 4 times with 25 ml of dimethylcarbonate. The residue was dried under vacuum at 30° C. under nitrogen atmosphere, for 1 hour.

### Example 8

### Preparation of Methyl Benzoate

0.04 mol (5 grams) of benzoic acid was suspended into a solution comprising 50 ml dimethylcarbonate. Subsequently, 0.018 mol of H₂SO₄ was dosed to the reaction mixture at T=20° C. increasing to T=84° C. The esterification reaction was proceeded for 18 hours at 84 to 85° C., after which the conversion into methylbenzoate was >99%.

### Example 9

### Preparation of (D)-phenylglycine methyl ester methanesulphonic acid salt

(PGM.CH₃SO₃H) at increased pressure and temperature. 100 g (D)-phenylglycine (ee_{D}>99) was suspended in a solution of 390 ml dimethyl-carbonate and 13.5 ml methanol. Subsequently, 90 ml methanesulphonic acid was added to the suspension in 30 to 60 min. After the dosage was completed, the reactor was closed and the temperature was increased to 103° C. with a concomittant increase of the pressure until 3 kg/cm². The reaction mixture was kept at these conditions for 15 hours while the pressure was maintained at 3 kg/cm² by adjusting the outlet of the CO₂ produced. The conversion of phenylglycine into phenylglycine methyl ester was >99.5% after 15 hours at 103° C. and 3 kg/cm². After completion of the reaction time, the overpressure was released and the mixture was cooled down to approximately 20° C. Subsequently, at 20° C., triethylamine was dosed to the reaction mixture to neutralize 85% of the excess of the methanesulphonic acid. The reaction mixture was filtered and the residue was washed with dimethylcarbonate. The yield of (D)-phenylglycine methyl ester was −90% (ee_{D}>99%).

### FIGURES

FIG. 1: IR Spectrum of DHMe.CH₃SO₃H

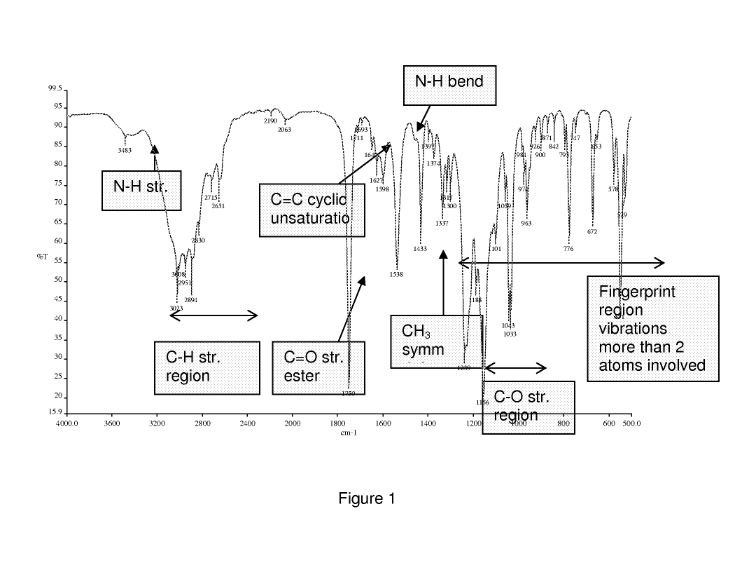

FIG. 2: IR Spectrum of PGM.CH₃SO₃H

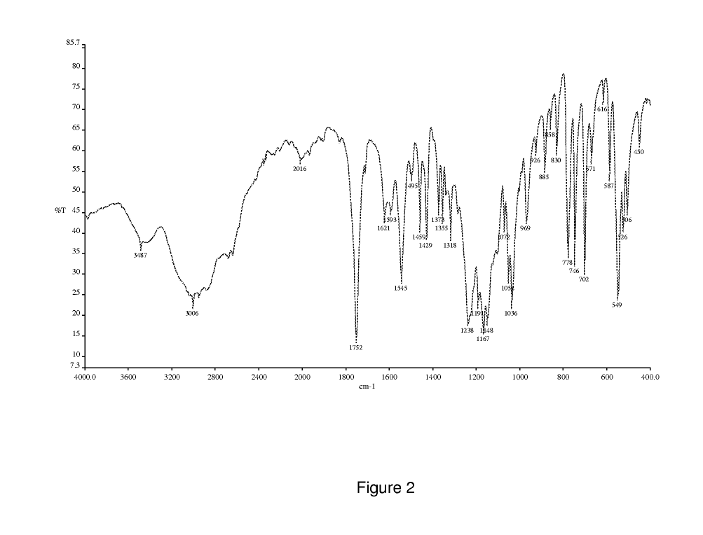

1. A. sulphonic acid salt of an amino acid alkyl ester.

2. A sulphonic acid salt according to claim 1, wherein the alkyl comprises 1 to 6 carbon atoms.

3. A sulphonic acid salt according to claim 1, wherein the amino acid is dihydro-phenylglycine or phenylglycine.

4. Process for the esterification of an organic acid into the corresponding organic acid ester comprising bringing the organic acid into contact with a strong acid and a solution comprising dialkylcarbonate in a reaction mixture.

5. Process according to claim 4, characterised in that the strong acid is selected from the group consisting of methane sulphonic acid, p-toluene sulphonic acid, benzene sulphonic acid and sulphuric acid.

6. Process according to claim 4 characterised in that the solution further comprises an alcohol, wherein the alcohol comprises a number of carbon atoms, which is identical to the number of carbon atoms of the alkyl in dialkylcarbonate.

7. Process according to claim 4, characterised in that the esterification comprises keeping the reaction mixture at a temperature below the reflux temperature of the dialkylcarbonate for at least 1 hour preferably for at least 4 hours.

8. Process according to claim 4, characterised in that the pressure is above atmospheric, preferably equal to or higher than 1 kg/m², more preferably at a pressure equal to or higher than 2 kg/m², even more preferably at a pressure equal to or higher than 3 kg/m².

9. Process according to claim 4, characterised in that the esterification further comprises keeping the reaction mixture at a temperature of at least
the reflux temperature of the dialkylcarbonate for at least 6 hours, preferably at least 24 hours, preferably between 48 hours and 7 days.

10. Process according to claim 4, further characterised by isolation of the organic acid ester from the reaction mixture.

11. Process according to claim 4, characterised in that the isolation of organic acid ester comprises adding a base to the reaction mixture.

12. Process according to claim 10, characterised in that the organic acid is an amino acid and the isolation comprises crystallising the amino acid ester in the form of a salt.

13. Use of a sulphonic acid salt of an amino acid alkyl ester in the synthesis of a β-lactam antibiotic.
